(19) United States
(12) Patent Application Publication
(10) Pub. No.: US 2022/0008380 A1
(43) Pub. Date: Jan. 13, 2022

(54) METHODS OF TREATING COVID-19 PATHOGENESIS

(71) Applicant: UT-Battelle, LLC, Oak Ridge, TN (US)

(72) Inventors: Daniel A. Jacobson, Oak Ridge, TN (US); Erica Teixeira Prates, Oak Ridge, TN (US); John I. Miller, Oak Ridge, TN (US); Michael R. Garvin, Oak Ridge, TN (US)

(21) Appl. No.: 17/368,911

(22) Filed: Jul. 7, 2021

Related U.S. Application Data
(60) Provisional application No. 63/048,709, filed on Jul. 7, 2020.

(51) Int. Cl. A61K 31/352; A61K 38/22; A61K 31/58; A61K 38/08; A61K 31/59; A61K 45/06

(52) U.S. Cl. CPC A61K 31/352; A61K 38/2292; A61K 45/06; A61K 38/08; A61K 31/59; A61K 31/58

(57) ABSTRACT

This disclosure provides methods for treating a SARS-CoV-2 infection in a subject comprising administering to the subject an effective amount of an inhibitor of COVID-induced RAS imbalance, that downregulates, for example, the Bradykinin system, the Renin-Angiotensin system, the hyaluronan synthesis pathway, or the fibrinogenesis pathway.

### CROSS REFERENCE TO RELATED APPLICATION

This application claims the benefit of priority from U.S. Provisional Application No. 63/048,709, filed Jul. 7, 2020, the entire contents of which are incorporated herein by reference.

### STATEMENT REGARDING FEDERALLY SPONSORED RESEARCH OR DEVELOPMENT

This disclosure was made with government support under a research project supported by Prime Contract No. DE-AC05-00OR22725 awarded by the U.S. Department of Energy. The government has certain rights in this invention.

### INCORPORATION BY REFERENCE OF SEQUENCE LISTING

The Sequence Listing in the ASCII text file, named as 39582_4671_1_SequenceListing.txt of 1 KB, created on Jun. 27, 2021, and submitted to the United States Patent and Trademark Office via EFS-Web, is incorporated herein by reference.

### BACKGROUND

The COVID-19 beta-coronavirus epidemic that originated in Wuhan, China in December of 2019 is now a global pandemic and is having devastating societal and economic impacts. The increasing frequency of the emergence of zoonotic viruses such as Ebola, Severe Acute Respiratory Syndrome (SARS), and Middle East Respiratory Syndrome (MERS) (among others) are of grave concern because of their high mortality rate (10%-90%). The cause of mortality appears to be heterogeneous and although it typically targets older individuals, younger individuals are also at risk. A key to combating the pandemic is to understand the molecular basis of COVID-19 that may lead to effective treatments.

### BRIEF SUMMARY OF THE DISCLOSURE

This disclosure provides a method for treating a SARS-CoV-2 infection in a subject comprising administering to the subject an effective amount of an inhibitor of COVID-induced imbalance of the renin angiotensis system (RAS).

In some embodiments, the COVID-induced imbalance of RAS is represented by increases in one or more of ACE2, renin, angiotensin, RAS receptors, kinogen, kallikrein enzymes that activate kinogen, bradykinin receptor B1, and bradykinin receptor B2.

In some embodiments, the inhibitor downregulates the Bradykinin system, the Renin-Angiotensin system, the hyaluronan synthesis pathway, or the fibrinogenesis pathway.

In some embodiments, the inhibitor is selected from a nucleic acid inhibitor, an inhibitory antibody, or a small molecule inhibitor.

In some embodiments, the inhibitor downregulates the Bradykinin system. In some embodiments, the inhibitor is a small molecule inhibitor selected from 2,3-Isoxazolethisterone, 17α-Methyl-2′H-5α-androst-2-eno[3,2-c]pyrazol-17β-ol, icatibant, ecallantide, or a C1 esterase inhibitor. In some embodiments, the inhibitor is a nucleic acid inhibitor of bradykinin.

In some embodiments, the inhibitor downregulates the Renin-Angiotensin system. In some embodiments, the inhibitor is an inhibitor of renin. In some embodiments, the inhibitor is selected from Vitamin D, pepstatin (isovaleryl-L-valyl-L-valyl-statyl-L-alanyl-statine), a peptide inhibitor of renin, or a non-peptide renin inhibitor.

In some embodiments, the inhibitor downregulates the hyaluronan synthesis pathway. In some embodiments, the inhibitor is 4-methylumbelliferone.

In some embodiments, the inhibitor downregulates the fibrinogenesis pathway. In some embodiments, the inhibitor is Thymosin beta-4.

### BRIEF DESCRIPTION OF THE FIGURES

FIGS. 1A-1B. Critically disrupted RAS and Bradykinin pathways in COVID-19 BAL samples. (A) Significantly differentially expressed genes: genes upregulated in COVID-19 (CYP3A4, CYP24A1, REN, AGT, ACE2, KNG1, KLK1, AGTR2, CPN1, and NOS1), blue are downregulated genes (VDR, ACE, SERPING1, NFKB1, MAPK1) scaled to the log₂-fold-change values for COVID-19. The overall effect is to shift the system to production of Ang and AGTR2-driven sensitization of BK receptors involved in pain (BDKRB1) and NO-dependent vasodilation (BDKRB2). Several points of inhibition maintain this imbalance. The suppression of NFkappaB by the virus decreases its binding to the ACE promoter and subsequent transcription. Decrease in the activation of Vitamin D and its receptor (VDR), which normally inhibits REN production, in combination with the upregulation of ACE2, increases flux of angiotensin to Ang₁₋₉ (top left). Decrease in the expression of the SERPING1 gene, lifts suppression of FXII of the intrinsic coagulation cascade, resulting in further production of BK from kallikrein and KNG (both upregulated) (top right). BK levels are further increased because ACE, which normally degrades it, is decreased. A surge in Ang₁₋₉ further sensitizes the effects of bradykinin at BDKRB2. Other enzymes that degrade BK are also downregulated such as MME, which is meant to degrade Ang₁₋₉, BK, and another important peptide Apelin (APLN). (B) The result of a hyperactive bradykinin system is vasodilation to the point of vascular leakage and infiltration of inflammatory cells.

FIG. 2. The upregulation of hyaluronan synthases and downregulation of hyaluronidases combined with the BK-induced hyperpermeability of the lung microvasculature leads to the formation of a HA-hydrogel that inhibits gas exchange in the alveoli of COVID-19 patients.

FIG. 3. Systemic-level effects of critically imbalanced RAS and BK pathways. The gene expression patterns from COVID BAL samples reveal a RAS that is skewed toward low levels of ACE that result in higher levels of Ang₁₋₉ and BK. High levels of ACE normally present in the lungs are responsible for generating system-wide angiotensin-derived peptides. The Bradykinin-Storm is likely to affect major organs that are regulated by angiotensin derivatives. These include altered electrolyte balance from affected kidney and heart tissue, arrhythmia in dysregulated cardiac tissue, neurological disruptions in the brain, myalgia in muscles and severe alterations in oxygen uptake in the lung itself. REN, ACE2, AGTR2 and the Bradykinin pathway are upregulated, whereas Vitamin D receptor and ACE are downregulated.

### DETAILED DESCRIPTION OF THE DISCLOSURE

Unless defined otherwise, all technical and scientific terms used herein have the same meaning as commonly understood by one of ordinary skill in the art to which this invention belongs.

As used herein, the term “about” refers to an approximately +/−10% variation from a given value.

### General Description

The entry point for the SARS-CoV-2 virus is ACE2, which is a component of the counteracting hypotensive axis of RAS, that produces the nonapeptide angiotensin1-9 (Ang₁₋₉) from angiotensin I. Bradykinin is a potent, but often forgotten, part of the vasopressor system that induces hypotension and vasodilation, and is regulated by ACE and enhanced by angiotensin 1-9. Analysis was performed on gene expression data from cells of bronchoalveolar lavage samples from COVID-19 patients that were used to sequence the virus, but the host information was discarded. Comparison with lavage samples from controls identify a critical imbalance in RAS represented by decreased expression of ACE in combination with increases in ACE2, renin (REN), angiotensin (AGT), key RAS receptors (AGTR2, AGTR1), kinogen (KNG) and the kallikrein enzymes (KLKB1, many of KLK-1-15) that activate it, and both bradykinin receptors (BDKRB1, BDKRB2). This very atypical pattern of the RAS is predicated to elevate bradykinin levels in multiple tissues and systems that will likely cause increases in vascular dilation, vascular permeability and hypotension. These bradykinin-driven outcomes explain many of the symptoms being observed in COVID-19. Furthermore, the inventors have integrated this mechanism with the overproduction of hyaluronic acid which forms a hydrogel in the lungs of COVID-19 patients that will prevent gas exchange.

### Methods of Treatment

Disclosed herein are methods for treating a SARS-CoV-2 infection in a subject comprising administering to the subject an effective amount of an inhibitor of COVID-induced imbalance of the renin angiotensis system (RAS).

In some embodiments, the COVID-induced imbalance of RAS is represented by increases in one or more of ACE2, renin, angiotensin, RAS receptors, kinogen, kallikrein enzymes that activate kinogen, bradykinin receptor B1, and bradykinin receptor B2. Such RAS imbalance is believed to elevate bradykinin levels in multiple tissues and systems that will likely cause increases in vascular dilation, vascular permeability and hypotension.

In accordance with this disclosure, an inhibitor of RAS imbalance refers to a molecule that acts upon a molecule in the renin angiotensin system (RAS), or in a related system or extension thereof including the bradykinin system, the hyaluronan synthesis pathway, and the fibrinogenesis pathway, so as to restore the balance of RAS. In various embodiments, the inhibitor is a molecule that downregulates the renin angiotensin system (RAS), the bradykinin system, the hyaluronan synthesis pathway, or the fibrinogenesis pathway, which are systems and pathways that may be elevated by COVID. By “downregulate” it is meant that an inhibitor inhibits, suppresses, or reduces the activity of a pathway by acting on a molecule involved in the pathway (e.g., ACE2, renin, angiotensin, RAS receptors, kinogen, kallikrein enzymes that activate kinogen, bradykinin receptor B1, and bradykinin receptor B2), for example, by inhibiting the level or activity of the molecule. The extent of downregulation can be at least 10%, 15%, 20%, 25%, 30%, 35%, or more of inhibition of the level or activity of a molecule, as compared to the level or activity of the molecule in the absence of the inhibitor.

In some embodiments, the method comprises administering to the subject an effective amount of a combination that comprises at least two inhibitors that inhibit at least two pathways. In some embodiments, the inhibitors in the combination act synergistically (i.e., the total therapeutic effect of the composition is greater than the sum of the therapeutic effects of the individual inhibitors). In some embodiments, the inhibitors in the combination of at least two inhibitors act additively (i.e., the total therapeutic effect of the composition is equal to the sum of the therapeutic effects of the individual inhibitors).

As used herein, the term “effective amount” refers to the amount of an inhibitor, or an active component of a pharmaceutical composition or method that is sufficient to show a meaningful patient benefit, i.e., treatment, healing, prevention of the relevant medical condition, amelioration of the symptoms, or an increase in rate of treatment, healing, prevention or amelioration of such conditions, or inhibition of the progression of the condition. When applied to an individual active ingredient, administered alone, the term refers to that ingredient alone. When applied to a combination, the term refers to combined amounts of the active ingredients that result in a desired therapeutic effect, whether administered in combination, serially or simultaneously.

In some embodiments, the inhibitor downregulates the Bradykinin system, the Renin-Angiotensin system, the hyaluronan synthesis pathway, or the fibrinogenesis pathway.

In some embodiments, the inhibitor is selected from a nucleic acid inhibitor, an inhibitory antibody, or a small molecule inhibitor.

In some embodiments, the inhibitor is a nucleic acid inhibitor as described herein. In some embodiments, the nucleic acid inhibitor is selected from the group consisting of an antisense RNA, a small interfering RNA, an RNAi, a microRNA, an artificial microRNA, and a ribozyme.

In some embodiments, the inhibitor downregulates the Bradykinin system.

In some embodiments, the inhibitor is a small molecule inhibitor selected from 2,3-Isoxazolethisterone (Danazol or DANOCRINE®) with the chemical formula:

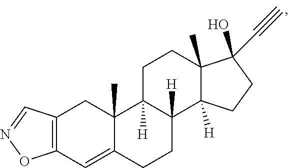

17α-Methyl-2′H-5α-androst-2-eno[3,2-c]pyrazol-17β-ol (Stanazolol) with the chemical formula:

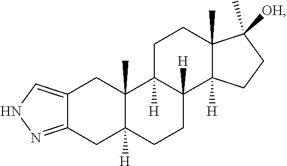

icatibant with the chemical formula:

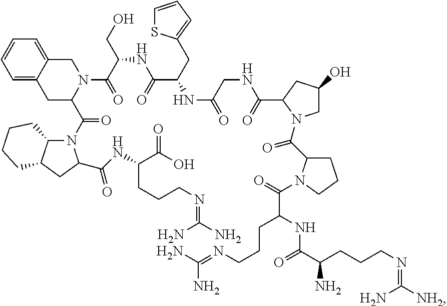 [SMILES: NC(N)=NCCCC(NC(=O)[C@H](N)CCCN=C(N)N)C(=O)N1CCCC1C(=O)N1C[C@H](O)CC1C(=O)NCC(=O)N[C@@H](Cc1cccs1)C(=O)N[C@@H](CO)C(=O)N1Cc2ccccc2CC1C(=O)N1C(C(=O)N[C@@H](CCCN=C(N)N)C(=O)O)C[C@@H]2CCCC[C@@H]21]

ecallantide, or C1 esterase inhibitor (e.g., BERINERT®, CINRYZE®, or HAEGARDA®).

In some embodiments, the inhibitor is a nucleic acid inhibitor of bradykinin. As used herein, the term “bradykinin” refers to a 9-amino acid peptide chain. The amino acid sequence of bradykinin is: Arg-Pro-Pro-Gly-Phe-Ser-Pro-Phe-Arg (RPPGFSPFR) (SEQ ID NO: 1).

In some embodiments, inhibitor downregulates the Renin-Angiotensin system.

In some embodiments, the inhibitor is a renin inhibitor.

In some embodiments, the inhibitor is selected from Vitamin D, pepstatin (isovaleryl-L-valyl-L-valyl-statyl-L-alanyl-statine), a peptide inhibitor of renin (e.g., a peptide analogue of renin, or a peptide analogue of the substrate angiotensinogen) or a non-peptide renin inhibitor.

In some embodiments, the non-peptide renin inhibitor is Remikiren with the following chemical formula:

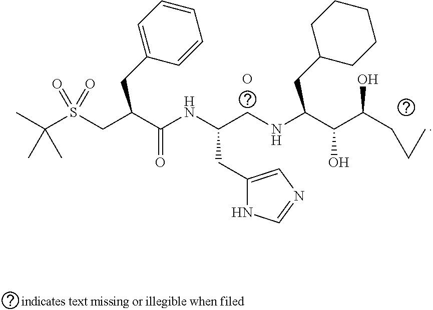

In some embodiments, the non-peptide renin inhibitor is Aliskiren with the following chemical formula:

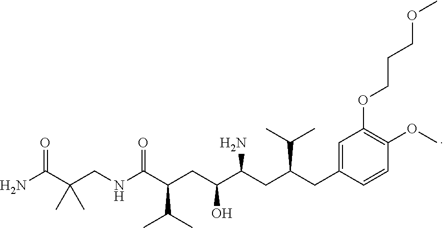 [SMILES: COCCCOc1cc(C[C@@H](C[C@H](N)[C@@H](O)C[C@H](C(=O)NCC(C)(C)C(N)=O)C(C)C)C(C)C)ccc1OC]

In some embodiments, the peptide inhibitor of renin is H-142 with the following chemical formula:

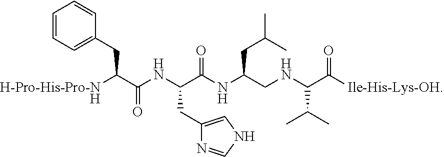

In some embodiments, the inhibitor downregulates the hyaluronan synthesis pathway.

In some embodiments, the inhibitor is 4-methylumbelliferone (4-MU or Hymecromone) with the following chemical formula:

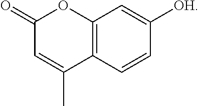

In some embodiments, the inhibitor downregulates the fibrinogenesis pathway.

In some embodiments, the inhibitor is Thymosin beta-4 (Timbetasin).

### Nucleic Acid Inhibitors

As used herein “nucleic acid inhibitor” refers to a nucleic acid that can reduce or prevent expression or activity of a target gene.

A number of nucleic acid-based methods, including antisense RNA, ribozyme directed RNA cleavage, post-transcriptional gene silencing (PTGS), e.g., RNA interference (RNAi), microRNA and artificial microRNA and transcriptional gene silencing (TGS) can be used to inhibit bradykinin gene expression in cells. Suitable inhibitors include full-length nucleic acids of allelic variants of bradykinin gene, or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid fragment from a gene to be repressed is cloned and operably linked to a heterologous regulatory region and a transcription termination sequence so that the antisense strand of RNA is transcribed. The recombinant vector is then transformed into plants, as described below and the antisense strand of RNA is produced. The nucleic acid fragment needs not be the entire sequence of the gene to be repressed, but typically is substantially complementary to at least a portion of the sense strand of the gene to be repressed. By “substantially complementary” it is meant that the nucleic acid fragment is capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, 8%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization.

In another method, a nucleic acid can be transcribed into a ribozyme or catalytic RNA, which affects expression of an mRNA. See, U.S. Pat. No. 6,423,885. Ribozymes can be designed to specifically pair with a target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes cleave mRNAs at locations dictated by flanking regions that form complementary base pairs with the target mRNA. See, for example, U.S. Pat. No. 5,254,678; Perriman et al., PNAS 92(13):6175-6179 (1995); de Feyter and Gaudron, Methods in Molecular Biology, Vol. 74, Chapter 43, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence or a fragment thereof, of the polypeptide of interest. The length of the sequence that is similar or identical to the sense coding sequence can be from 10 nucleotides to 500 nucleotides, from 15 nucleotides to 300 nucleotides, from 20 nucleotides to 100 nucleotides or from 25 nucleotides to 100 nucleotides. The other strand of the stem portion of a double stranded RNA comprises a sequence that is similar or identical to the antisense strand or a fragment thereof, of the coding sequence of the polypeptide of interest and can have a length that is shorter, the same as or longer than the corresponding length of the sense sequence. In some cases, one strand of the stem portion of a double stranded RNA comprises a sequence that is similar or identical to the 3′ or 5′ untranslated region or a fragment thereof, of the mRNA encoding the polypeptide of interest and the other strand of the stem portion of the double stranded RNA comprises a sequence that is similar or identical to the sequence that is complementary to the 3′ or 5′ untranslated region, respectively or a fragment thereof, of the mRNA encoding the polypeptide of interest. In other embodiments, one strand of the stem portion of a double stranded RNA comprises a sequence that is similar or identical to the sequence of an intron or a fragment thereof in the pre-mRNA encoding the polypeptide of interest and the other strand of the stem portion comprises a sequence that is similar or identical to the sequence that is complementary to the sequence of the intron or fragment thereof in the pre-mRNA.

A construct including a sequence that is operably linked to a heterologous regulatory region and a transcription termination sequence and that is transcribed into an RNA that can form a double stranded RNA, can be transformed into plants as described below. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330 and 20030180945.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA or an intron in a pre-mRNA encoding a polypeptide of interest or a fragment of such sequences. In some embodiments, the sense or antisense sequence is identical or complementary to a sequence of the regulatory region that drives transcription of the gene encoding a polypeptide of interest. In each case, the sense sequence is the sequence that is complementary to the antisense sequence.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence. The two regulatory regions can be the same or different. The two transcripts can form double-stranded RNA molecules that induce degradation of the target RNA. In some cases, a nucleic acid can be positioned within a P-DNA such that the left and right border-like sequences of the P-DNA are on either side of the nucleic acid.

In some embodiments, a suitable nucleic acid inhibitor can be a nucleic acid analog. Nucleic acid analogs can be modified at the base moiety, sugar moiety or phosphate backbone to improve, for example, stability, hybridization or solubility of the nucleic acid. Modifications at the base moiety include deoxyuridine for deoxythymidine and 5-methyl-2′-deoxycytidine and 5-bromo-T-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite or an alkyl phosphotriester backbone.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The present disclosure is further illustrated by the following non-limiting examples.

### EXAMPLES

### Example 1: Materials and Methods

Gene Expression Analysis:

FASTQ files were downloaded from the Sequence Read Archive (PRJNA605983 and PRJNA434133) on NCBI and trimmed using the default parameters in CLC Genomics Workbench (20.0.3). RNA-Seq analysis was performed using the latest version of the human transcriptome (GRCh38_latest_rna.fna, 160,062 transcripts to which were appended the SARS-CoV-2 reference genome, MN908947). Mapping parameters were set with a mismatch cost of two, insertion and deletion cost of three, and both length and similarity fraction were set to 0.85. TPMs were generated fir all 160,063 transcripts for the nine COVID-19 samples and the 40 controls. The resulting transcript mappings for genes of interest were manually inspected to account for any expression artifacts, such as reads mapping solely to repetitive elements such as the Alu transposable element or all reads mapping to a UTR or pseudogene therein. Transcripts whose counts came solely from (or were dominated by) reads at repetitive elements were removed from the analysis. For the controls an outlier analysis was run using the prcomp function in the R package factoextra. Input data were TPM for transcripts that averaged greater than one across all samples.

To test the hypothesis that gene transcripts were differentially expressed in the COVID-19 patients vs controls, the edgeR package was used. Briefly, normalization factors were determined and the count data were scaled to account for library size according to the package instructions. Then, dispersion was estimated and a negative binomial model was fit to the read counts for each gene. Gene wise tests were then performed to test for differential expression. As described below, manual inspection of isoforms determined when there was isoform switching and differential isoform expression was calculated as described above. The p-values were adjusted for multiple comparisons using the Benjamini-Hochberg method.

Generation of Heat Maps:

Z-scores were calculated (from TPMs) for each of the differentially expressed genes across COVID-19 individuals and controls from the asthma study. The values were converted into a clustered heat-map using Seaborn Python script.

Transcript-vs. Gene-Level Analysis:

For each of the differentially expressed genes, transcript-level TPMs were plotted across COVID-19 and control individuals for visual inspection and annotation. Transcripts were collapsed to the gene-level if (1) all but one of the transcripts had low TPM, (2) different transcripts coded for the same protein, (3) and none of the transcripts were substantially truncated or otherwise altered in any functional domains.

Annotation Network Generation:

A gene set from the RAS and BK pathways was extracted from log₂ transformed GTEx expression lung data. Pearson correlation values were calculated among these genes and the resulting values clustered using hierarchical and k-means clustering of both genes and samples to identify patterns. Four k-means was sufficient to partition all of the genes. Two of the four clusters were highly anti-correlated: AGTR1 identified one pattern and AGTR2 identified an anti-correlated cluster. One of the two remaining intermediate clusters was partially correlated to and was subsequently merged with the AGTR1 cluster and the other remaining cluster was partially correlated to and therefore was merged with AGTR2 cluster. The resulting two clusters were annotated with functional terms and cell types in order to create the annotation network.

H-MAGMA Analysis:

A chromosomal coordinate for each VDR binding site was used to test for chromatin contact with RAS-BK genes of interest using H-MAGMA, which allows integration of chromatin interaction data for gene-set analysis. Each coordinate within a VDR binding site was assigned a population size of 500,000 and a p-value of 1×10⁻³⁵. The gene annotation file mapped all coordinates to genes that were either in the exonic or promoter region of said gene, or within a related chromatin region in intronic and intergenic regions. H-MAGMA returned a list of genes in contact with the coordinates of VDR binding sites. H-MAGMA identified six RAS-BK genes of interest, namely DPP4, BDKRB2, KLK6, KLK7, KLK10, and IKBKG.

### Example 2: The Renin Angiotensin System (RAS)

Nine bronchoalveolar lavage (BAL) samples were originally collected from patients in Wuhan China for RNA sequencing in order to determine the etiological agent for COVID-19 and resulted in the sequence of the first SARS-CoV-2 viral genome. However, the human reads from these samples were discarded. The inventors analyzed the human RNA-seq data from these BAL samples alongside 40 controls.

Although pre-existing hypertension is a reported comorbidity for COVID-19, recent reports indicate hypotension is highly associated with COVID-19 patients once in the hospital. The RAS is an important pathway linked to these conditions because it maintains a balance of fluid volume and pressure using several cleavage products of the peptide angiotensin (AGT) and their receptors. The most well studied peptide is angiotensin II (Ang II), which typically generates vasoconstriction and sodium retention via the AGTR1 receptor and vasodilation and natriuresis when binding to the AGTR2 receptor. The RAS also includes several other lesser-known peptides that are highly important; Ang₁₋₇ binds to the MAS 1 receptor, generating anti-inflammatory and vasodilatory effects, and Ang 1-9 binds to the AGTR2 receptor. Ang II is produced by the enzyme ACE whereas Ang 1_7 is generated by the combination of ACE and ACE2 activity and Ang₁₋₉ by ACE2 alone. It is important, therefore, to consider all of these components in the context of the others and not any one in isolation.

ACE2 is also the main receptor for the SARS-CoV-2 virus and is not highly expressed in normal lung tissue based on the Genotype-Tissue Expression (GTEx, website) population). However, results from the differential gene expression analysis of RAS genes in cells taken from BAL samples from individuals presenting with severe symptoms of COVID-19 demonstrates upregulation of ACE2 (199 fold) and disruption of this system compared to controls. In the COVID-19 samples, AGT (34 fold) and the enzyme that activates it (REN, 380 fold) are increased compared to controls whereas the enzymes that produce most of the cleavage products, including ACE (−8 fold), are downregulated, which will likely result in a shift of the entire RAS to produce Ang₁₋₉. In addition, the AGTR1 (430 fold) and AGTR2 (177 fold) receptors are upregulated in BAL COVID-19 samples.

Given the central role that the angiotensin and bradykinin (BK) peptides play in COVID-19 based on the gene expression analysis from BAL samples, the inventors next focused on the RAS- and BK-related gene pathways in lung tissue from the GTEx population; specifically the networks of genes that are correlated and anti-correlated with the expression of the angiotensin receptors AGTR2 and AGTR1. This subset of genes was annotated with functional information and cell type involvement which resulted in a network that, as would be expected, demonstrates their extensive involvement in arterial and vascular resistance and blood flow via microvascular dilation, flow, and fluid balance. The genes on the left side of the network are extensively involved in vasoconstriction and contain, among others, ACE, AGTR1, BDKR2, Nitric Oxide Synthase-1, and -2 (NOS1 and NOS2). The right side of the network is extensively involved in decreased arteriolar resistance (vasodilation), increased vascular permeabilization, and altered fluid balance and includes, among other genes, ACE2, AGTR2, and the Vitamin D Receptor (VDR). Surprisingly, the inventors find that both sides of the network are also clearly involved in immune system modulation.

### Example 3: The Bradykinin System

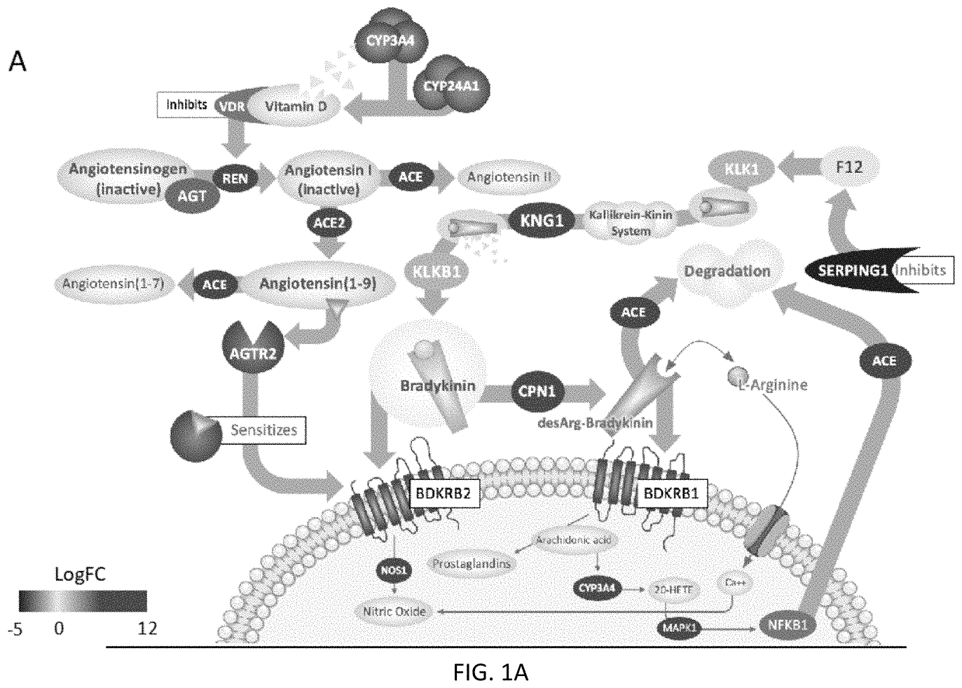

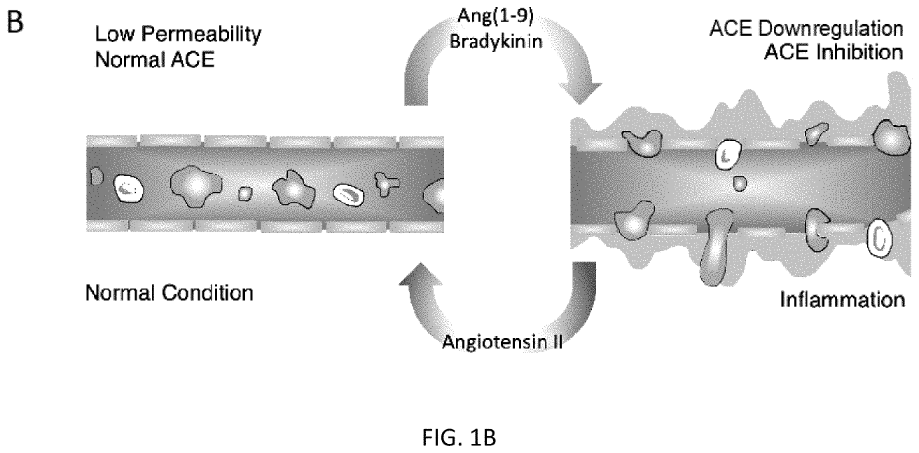

Although not as widely discussed as angiotensin, BK is another potent regulator of blood pressure and can be considered essentially an extension of the RAS. Briefly, similar to the angiotensin peptides, BK is produced from an inactive pre-protein kininogen (either circulating—HMWK or tissue—LWMK) through activation by the serine protease kallikrein (FIG. 1A). Kallikrein is represented by a cluster of serine proteases (KLK1-KLK15) on chromosome 19 with different tissue distributions; KLKB1 (on chromosome 4) is normally expressed in the pancreas and is responsible for circulating (plasma) kallikrein. These proteases are inactivated by zinc and several are known co-receptors for viruses including influenza. KLKB1 is activated by FXII of the intrinsic coagulation pathway, which is normally kept in check by the C1-Inhibitor encoded by SERPING1 (FIG. 1A). This has the vital ancillary effect of inhibiting the feedback loop of FXII activation by kallikrein.

Similar to AGTR2 stimulation, BK induces vasodilation, natriuresis, and hypotension upon activation of the BDKRB2 receptor. BK is tightly integrated with the RAS as BK receptor signaling is augmented by Ang₁₋₉, likely by resensitization of the BDKRB2 receptor and also because ACE degrades and inactivates BK. Interestingly, ACE has a higher affinity for BK than it does for AGT and therefore under conditions where ACE is low, the vasopressor system is tilted toward a BK-directed hypotensive axis (FIG. 1A). In addition to its role in pressure and fluid homeostasis, BK is a normal part of the inflammatory response after injury and acts to induce pain via stimulation of the BDKRB1 receptor by BK₁₋₈, which also causes neutrophil recruitment and increases in vascular permeability (FIG. 1B). BK₁₋₈ is produced by the enzyme carboxypeptidase N (CPN1 671 fold) acting on BK.

As with the RAS, the BK system is also severely affected in the COVID-19 BAL samples. The expression of the BK precursor kininogen and nearly all of the kallikreins are undetected in controls but expressed in COVID-19 BAL (FIG. 1A). Most of the enzymes that degrade BK, including ACE, are downregulated (−8 fold) in COVID-19 BAL compared to controls, directing BK₁₋₉ and BK₁₋₈ to the upregulated receptors BKB2R (207 fold) and BKB1R (2945 fold), respectively. Of note, the pain-receptor BKB1R is normally tightly controlled and expressed only at very low levels in nearly all tissues in GTEx, but in the case of COVID-19 BAL, both BK receptors are expressed whereas they are virtually undetected in controls. Finally, F12 is unchanged but the SERPING1 (−33 fold) gene that encodes the C1-Inhibitor that inhibits FXII is highly down-regulated, which would result in even further increases in BK in COVID-19 patients given its role in KLKB1 (3 fold) activation. As described below, the resulting Bradykinin Storm is likely responsible for most of the observed COVID-19 symptoms.

### Example 4: Hyaluronic Acid Synthesis and Degradation

Hyaluronic acid (HA) is a polysaccharide found in most connective tissues. HA can trap roughly 1000 times its weight in water and when bound to water the resulting hydrogel obtains a stiff viscous quality similar to “Jello.” HAS1, HAS2 and HAS3 are genes that encode hyaluronan synthases which are integral membrane proteins responsible for HA production. HA is degraded by hyaluronidases encoded by HYAL1 and HYAL2. Proteins encoded by other genes in this family (HYAL3 and HYAL4) do not appear to have a hyaluronidase activity. HYAL1 encodes a lysosomal hyaluronidase (Hyal-1) active at low pH and is responsible for intracellular degradation of HA. HYAL2 encodes a membrane-bound hyaluronidase (Hyal-2) responsible for extracellular degradation of HA. Both Hyal-1 and Hyal-2 are dependent on CD44 (an HA receptor) for activity.

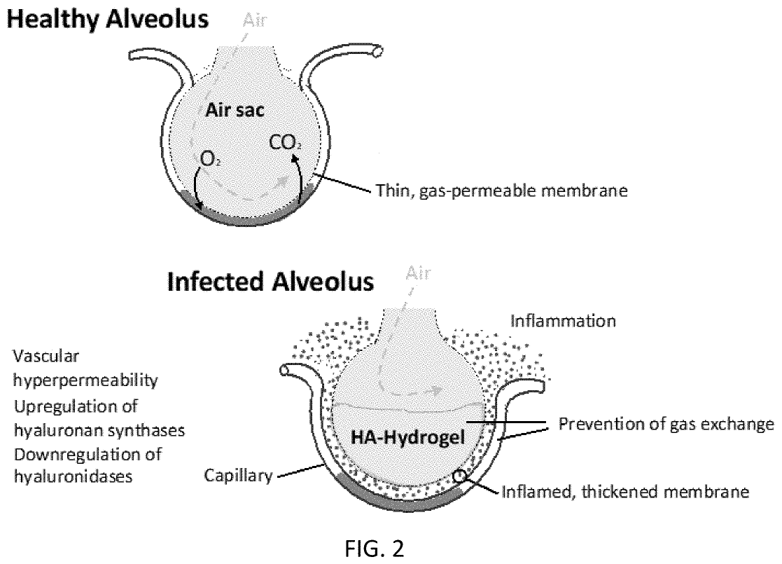

As with the RAS and BK systems, the genes encoding HA synthesis and degradation are also severely affected in the COVID-19 BAL samples. There is significant upregulation of the genes involved in HA synthesis: HAS1 (9113-fold), HAS2 (493 fold), and HAS3 (32 fold). The CD44 gene that encodes the HA receptor required for HA degradation and the gene encoding extracellular hyaluronidase HYAL2 are both downregulated (−11 and −5 fold respectively) in the BAL fluid of COVID-19 patients. HYAL1 is not expressed in the BAL fluid of controls or the COVID-19 patients. The result of these shifts in expression would be likely to cause an increase in the amount of HA in the bronchoalveolar space of the lungs which, combined with the vascular hyperpermeability caused by BK, could form a viscous hydrogel that would negatively impact gas exchange (FIG. 2). In fact, HA in BAL fluid has previously been associated with acute respiratory distress syndrome (ARDS) where there was a significant anticorrelation between the concentration of HA and the pulmonary oxygenation index. HA has also been associated with pulmonary thrombosis and/or ground glass opacities in radiological findings.

Although not the focus of the present study, coagulopathy is commonly reported in cases of COVID-19 and there are suggestions in the literature of links between RAS and coagulopathy. The Ang₁₋₉ peptide that is increased in COVID-19 BAL stimulates thrombosis by inhibiting fibrinolysis. In addition to BK, ACE also degrades the antifibrotic peptide N-acetyl-seryl-aspartyl-lysyl-proline (AcSDKP), which is produced from thymosin beta-4 (TMSB4X, −130 fold). Increased fibrinolysis could therefore be achieved by increasing ACE, or by administering thymosin beta-4, which is currently in clinical trials for the treatment of cardiovascular disorders (Timbetasin). If TMSB4X is, in fact, protective, it could explain the lower incidence of COVID-19 induced mortality in women because it is found on the X chromosome and escapes X-inactivation. Women therefore would have twice the levels of this protein than men, which is supported by the BAL analysis (−207 fold in males, −131 fold in females).

In addition, both the RAS and BK pathways have previously been tied to HA. It was found that Angiotensin II increased CD44 expression and hyaluronidase activity. As discussed above, COVID-19 likely significantly downregulates the production of Angiotensin II which is consistent with the decrease in CD44 expression that is seen in the BAL fluid of SARS-CoV-2 infected patients. Furthermore, IL2 was recently reported to be highly upregulated in symptomatic but not asymptomatic COVID-19 patients and is upregulated (21 fold) in the BAL samples compared to controls. This cytokine is induced by BK in the lung, causes vascular leakage syndrome (VLS), which appears to be mediated through CD44. Interestingly, CD44 knockout mice displayed reduced IL2-induced VLS, suggesting this may be a valuable target for COVID-19 intervention.

### Example 5: Clinical Description of COVID-19

According to the CDC, the majority of SARS-CoV-2 infections are asymptomatic or mild. Those that proceed to more severe forms present with fever, a non-productive cough that may result in hemoptysis and shortness of breath. Other common symptoms are myalgia, fatigue, sore throat, nausea, vomiting, diarrhea, conjunctivitis, anorexia, and headache (the CDC website). Reports from blood studies include leukopenia, eosinopenia, neutrophilia, elevated liver enzymes, C-reactive protein, and ferritin. Furthermore, autopsies have reported extensive hyaline membrane formation in the lungs of COVID-19 patients. Specifically, histological analysis of the lungs of a deceased COVID-19 patient showed organizing hyaline membranes in the early stages of alveolar lesions and prominent hyaline membranes in the exudative phase of diffuse alveolar damage. In a separate postmortem study of lung tissue from COVID-19 patients, microscopic examination found “numerous hyaline membranes without evidence of interstitial organization.” Furthermore, in another autopsy study of a COVID-19 patient, histological analysis found extensive hyaline membranes, which the authors interpreted as indicative of ARDS. Finally, a meta-analysis showed that there was a statistically significant 4.6-fold difference in lung weight of COVID-19 patients versus controls, which they conclude is consistent with the HA-hydrogel formation known to occur in ARDS.

Although much focus has been on the lung due to the need for ventilator support of end-stage disease, COVID-19 also affects the intestine, liver, kidney, heart, brain, and eyes. Nearly one-fifth of hospitalized patients experience cardiac injury, many of whom have had no history of cardiovascular problems prior to infection. Responses include acute myocardial injury, myocarditis, and arrhythmias that may be due to viral infection directly, which is consistent with high expression of the SARS-CoV-2 receptor ACE2 in cardiac tissue (gtexportal website). An important extension of the RAS in controlling cardiac contraction and blood pressure is the potent inotrope apelin (APLN), which acts as an NO-dependent vasodilator when its receptor (APLNR) heterodimerizes with BDKRB1. APLN (98 fold), APLNR (3190 fold) and BDKRB1 (2945 fold) are all upregulated in COVID-19 BAL. As with BK and ANG derived peptides, APLN is inactivated by Neprilysin (MME), which is significantly downregulated in the BAL samples from COVID-19 individuals (−16 fold). Therefore, increased APLN-signaling can be added to the imbalanced RAS.

In addition to cardiac dysfunction, neurological involvement in COVID-19 was revealed after an MRI assessment of COVID-19-positive patients with encephalopathy symptoms in France identified enhancement in leptomeningeal spaces and bilateral frontotemporal hypoperfusion which are consistent with increased vascular permeabilization in the brain. Furthermore, earlier reports from China indicate high frequencies of dizziness, headache, as well as taste and smell impairment. The most recent reports from the United States and China indicate that 30-50% of COVID-19 patients experience adverse gastrointestinal symptoms. Direct infection by the virus and damage to the kidney was also observed, specifically in the proximal tubules. These latter two findings are not surprising given the higher expression of ACE2 in these tissues compared to tissues overall (gtexportal website), which would facilitate infection by the virus. Finally, COVID-19 patients also frequently display skin rashes including “covid-toe” that appear to be related to dysfunction of the underlying vasculature.

### Example 6: Bradykinin Storms: A Model of SARS-CoV-2, COVID-19, and BK-Driven Vascular Permeabilization

Based on previous work in SARS-CoV-1 and SARS-CoV-2, it is likely that this new coronavirus enters host cells in nasal passages where the receptor ACE2 is moderately expressed. Migration to throat tissues and passage through the stomach is then possible given that SARS-CoV-2 can survive the extreme pH of the gastric tissues and infection could then expand into the intestines where ACE2 levels are high. Initial infection might not occur in the lung epithelium given that ACE2 is undetectable or expressed at extremely low levels there. Following infection, the single polypeptide that is translated from the virus' positive-strand RNA genome is cleaved into active proteins by the non-structural protein 3CL^{pro} protease. This protein is then repurposed by the virus to inactivate the host cells' first line of defense, interferon, most likely by degrading the NFkappaB activating factor IKK-gamma as has been shown to happen in the porcine coronavirus PEDV.

Aside from self-protection, the suppression of NFkappaB (−9 fold reduced in BAL samples) directly affects the RAS as NFkappaB normally induces the expression of ACE by binding to its promoter and increasing transcription (FIG. 1A). This likely relates to the role of ACE in the innate immune response that is independent of its actions on the vascular system. The virus therefore acts pharmacologically as an ACE inhibitor by reducing its RNA expression more than 10-fold, which is supported by the instant BAL RNA-seq analysis. Additionally, ACE2 expression is normally downregulated in-part by Ang II. As Ang II is the catalytic product of ACE it would seem that the virus's ability to decrease ACE expression would have the effect of upregulating ACE2 (199 fold in the instant BAL analysis). In some patients, severe pulmonary involvement could occur when the virus is introduced into the intestinal lymph vessels and moves up the lymphatic system, enters the bloodstream at the thoracic duct and moves through the heart and into the lung microvasculature where it could attack cells in the lungs that now express ACE2 due to virus-induced upregulation.

Given that the high levels of ACE in the vascular bed of the lung are the major producer for circulating angiotensin-derived peptides, establishment of SARS-CoV-2 in the lung will have profound effects. Downregulation of ACE here (confirmed in BAL samples from COVID-19 patients) will result in the diversion of the RAS to produce the BK-augmenting peptide Ang₁₋₉, exacerbating BK-effects, such as pain sensitization and increased vascular permeability on a system-wide level. Expansion of this imbalance as described above (FIGS. 1A-1B), increases levels of BK and will result in increased vascular permeability in tissues that have been infected by SARS-CoV-2 and be most severe in those that are normally regulated by ACE. ACE may also provide a key diagnostic point as half of the variation amongst individuals can be explained by an insertion/deletion polymorphism of the gene.

As mentioned above, the combination of vascular permeability and HA build up in the lungs could produce a hydrogel that significantly inhibits gas exchange in bronchoalveolar spaces. This is consistent with the autopsy reports of hyaline membranes in the lungs of deceased COVID-19 patients as well as other acute respiratory distress conditions (e.g. SARS, MERS, ARDS). Although this likely represents a late-stage event in severe cases of COVID-19, if the cause is overproduction of HA as a result of disruption of the RAS, it is also a potentially valuable intervention point because the condition is easily identified, and treatment could have rapid and significant beneficial effects.

In addition, increased levels of the vasodilating peptide APLN that are produced in COVID-19 patients could have spillover effects on cardiac function. APLN upregulates the expression of ACE2 and directly affects cardiac contraction and vasodilation. Increased levels of APLN are known to be associated with cardiac arrhythmia and in the case of hyper-stimulated BK output, could be causing cardiac events in COVID-19 patients. In addition, increased levels of APLN could lead to more ACE2 receptors for SARS-CoV-2 in the heart and thus stimulate further infection.

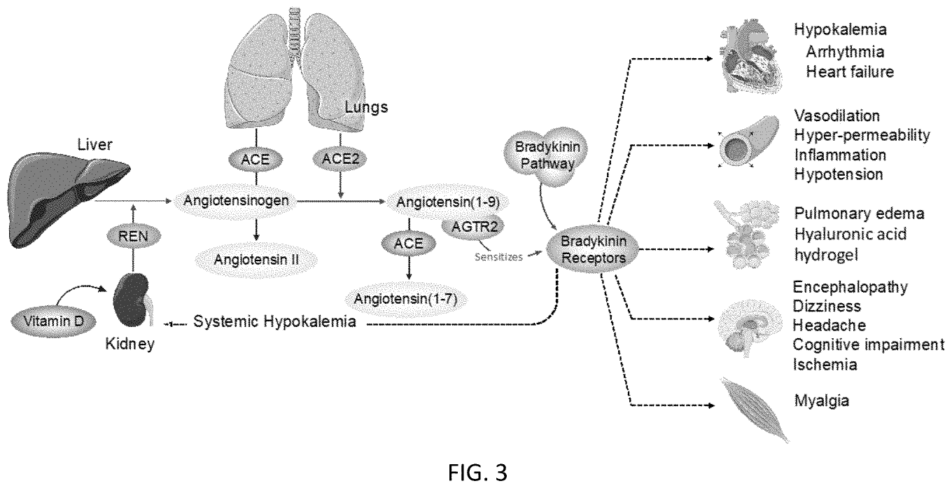

Furthermore, excess BK can lead to hypokalemia which is associated with arrhythmia and sudden cardiac death, both of which have been reported in COVID-19 patients; a recent report confirms that hypokalemia is occurring in severe cases of COVID-19. It is also notable that many of the other symptoms being reported for COVID-19 (myalgia, fatigue, nausea, vomiting, diarrhea, anorexia, headaches, decreased cognitive function) are remarkably similar to other hyper-BK-conditions that lead to vascular hyper-permeabilization such as angioedema as was recently noted. In agreement with that report, instant results indicate that the pathogenesis of COVID-19 is likely the result of Bradykinin Storms rather than cytokine storms (although given the induction of IL2 by BK, the two may be intricately linked). This model predicted that a loss of ACE2 would exacerbate the BK-induced pathogenesis. However, the BAL fluid expression data indicates that the Bradykinin Storm is instead caused by upregulation of ACE2 and reduced degradation of BK by ACE. Based on this data-driven model, an individual's symptomatology is likely directly related to the specific tissue distribution of viral infection around the body (FIG. 3) and should be viewed in the context of an overactive bradykinin response. The majority of circulating BK is degraded by ACE in the lung and therefore heterogeneous symptoms of COVID-19 could also be the result of systemic effects of increased levels of circulating bradykinin and the 8-fold reduction of ACE in the lung microvasculature that would normally degrade it.

Given this model, factors that affect RAS balance should be further investigated in the framework of diagnosis and treatment. For example, another well documented regulator of RAS is Vitamin D as the liganded Vitamin D receptor (VDR) suppresses REN expression. Patients who are deficient in Vitamin D are at-risk for ARDS in general and Vitamin D deficiencies have recently been associated with severity of illness in COVID-19 patients. The instant BAL gene expression analysis shows that VDR is 2-fold down-regulated and enzymes [CYP24A1 (465 fold), CYP3A4 (208 fold)] that catabolize Vitamin D (1.25(OH)2D) and its precursor (25 OHD) are up-regulated in COVID-19 patients compared to controls, which will likely result in further increases in REN. Furthermore, the inventors' analysis of ChipSeq experiments from a VDR study have determined that, in addition to REN, the following genes in the RAS-Bradykinin system have a VDR binding site within 20 kilobases: BDKRB1, BDKRB2, CYP24A1, DPP4, IKBKG (regulates NFkappaB), KLK1, KLK2, KLK4, KLK6, KLK7, KLK9, KLK10, and MME. Six of these binding sites can be tied to the following genes via chromatin structure with the use of H-MAGMA and Hi-C data (see Example 1: Methods): DPP4, BDKRB2, KLK6, KLK7, KLK10, and IKBKG. VDR binds to many sites in the genome with tissue-specific binding patterns, indicating putative associations to other genes in the RAS and BK pathways.

### Example 7: Potential Interventions

Several interventional points (most of them already FDA-approved pharmaceuticals) could be explored with the goal of increasing ACE, decreasing BK, or blocking BK2 receptors (Table 1). Icatibant is a BKB2R antagonist whereas Ecallantide acts to inhibit KLKB1, reducing levels of BK production. Androgens (danazol and stanasolol) increase SERPING1, although the side effects likely make these undesirable, but recombinant forms of SERPING1 (Berinert/Cinryze/Haegarda) could be administered to reduce BK levels. It should be noted that any intervention may need to be timed correctly given that REN levels rise on a diurnal cycle, peaking at 4 AM which corresponds with the commonly reported worsening of COVID-19 symptoms at night. Another approach would be the modulation of REN levels via Vitamin D supplementation when warranted. 4-methylumbelliferone (Hymecromone) is a potent inhibitor of HAS1, HAS2 and HAS3 gene expression and results in the suppression of the production of hyaluronan in an ARDS model. Hymecromone (4-methylumbelliferone) is approved for use in Asia and Europe for the treatment of biliary spasm. However, it can cause diarrhea with subsequent hypokalemia, so considerable caution should be used if this were to be tried with COVID-19 patients. As mentioned above, Timbetasin may reduce COVID-19 related coagulopathies by increasing fibrinolysis.

The testing of any of these pharmaceutical interventions should be done in well-designed clinical trials. Given the likely future outbreaks of zoonotic viruses with a similar outcome, it would be in the best interest long-term to invest in the development of small molecules that can inhibit the virus from replicating or suppressing the host immune system such as a 3CL^{pro} inhibitor. However, to date, no large multi-centered, randomized, placebo controlled, blinded clinical trials have been done with 3CL^{pro} inhibitors. In the meantime, the instant analyses suggest that prevention and treatment centered on vascular hyper-permeability and the suppression of hyaluronan may prove beneficial in fighting the pathogenesis of COVID-19. Inventors note that recent studies have validated the inventors' model's predictions of hypokalemia (Lippi G. et al., Ann. Clin. Biochem. 4563220922255 (2020)) and Vitamin D deficiency (Alipio M., SSRN Electronic Journal doi:10.2139/ssrn.3571484) in COVID-19 patients.

TABLE 1
Potential therapeutic interventions,
their targets, and predicted effect.

| Drug | Target | Predicted Effect |
|---|---|---|
| Danazol, Stanasolol | SERPING1 | Reduce Bradykinin |
| | | production |
| Icatibant | BKB2R | Reduce Bradykinin |
| | | signaling |
| Ecallantide | KLKB1 | Reduce Bradykinin |
| | | production |
| Berinert, Cinryze, Haegarda | SERPING1 | Reduce Bradykinin |
| | | production |
| Vitamin D | REN | Reduce Renin |
| | | production |
| Hymecromone | HAS1, HAS2, | Reduce hyaluronan |
| | HAS3 | |
| Timbetasin | TMSB4X | Increase fibrinolysis |

What is claimed is:

1. A method for treating a SARS-CoV-2 infection in a subject comprising administering to the subject an effective amount of an inhibitor of COVID-induced imbalance of the renin angiotensis system (RAS).

2. The method of claim 1, wherein the COVID-induced imbalance of RAS is represented by increases in one or more of ACE2, renin, angiotensin, RAS receptors, kinogen, kallikrein enzymes that activate kinogen, bradykinin receptor B1, and bradykinin receptor B2.

3. The method of claim 1, wherein the inhibitor downregulates the Bradykinin system, the Renin-Angiotensin system, the hyaluronan synthesis pathway, or the fibrinogenesis pathway.

4. The method of claim 1, wherein the inhibitor is selected from a nucleic acid inhibitor, an inhibitory antibody, or a small molecule inhibitor.

5. The method of claim 1, wherein the inhibitor downregulates the Bradykinin system.

6. The method of claim 5, wherein the inhibitor is a small molecule inhibitor selected from 2,3-Isoxazolethisterone, 17α-Methyl-2′H-5α-androst-2-eno[3,2-c]pyrazol-17β-ol, icatibant, ecallantide, or a C1 esterase inhibitor.

7. The method of claim 5, wherein the inhibitor is a nucleic acid inhibitor of bradykinin.

8. The method of claim 1, wherein the inhibitor downregulates the Renin-Angiotensin system.

9. The method of claim 8, wherein the inhibitor is an inhibitor of renin.

10. The method of claim 8, wherein the inhibitor is selected from Vitamin D, pepstatin (isovaleryl-L-valyl-L-valyl-statyl-L-alanyl-statine), a peptide inhibitor of renin, or a non-peptide renin inhibitor.

11. The method of claim 1, wherein the inhibitor downregulates the hyaluronan synthesis pathway.

12. The method of claim 11, wherein the inhibitor is 4-methylumbelliferone.

13. The method of claim 1, wherein the inhibitor downregulates the fibrinogenesis pathway.

14. The method of claim 13, wherein the inhibitor is Thymosin beta-4.


SEQUENCE LISTING (1 sequence)

SEQ ID NO: 1 - protein, 9 aa, Homo sapiens
RPPGFSPFR